(19) United States
(12) Patent Application Publication
(10) Pub. No.: US 2008/0053195 A1
(43) Pub. Date: Mar. 6, 2008

(54) Soot Generator

(75) Inventors: Ulrich Matter, Baden (CH); Thomas Mosimann, Meisterschwanden (CH); Markus Kasper, Widen (CH); Michael Riner, Villmergen (CH)

Correspondence Address: RANKIN, HILL & CLARK LLP, 38210 Glenn Avenue, WILLOUGHBY, OH, 44094-7808, US

(73) Assignee: MATTER ENGINEERING AG, Wohlen (CH)

(21) Appl. No.: 11/571,911

(22) PCT Filed: Jul. 7, 2005
(86) PCT No.: PCT/CH05/00390

§ 371 Date: Aug. 13, 2007

(30) Foreign Application Priority Data
Jul. 12, 2004 (EP) 04405441.9

(51) Int. Cl. G01N 33/00; B01J 12/00; C09C 1/60

(52) U.S. Cl. 73/28.01; 23/314; 422/150

(57) ABSTRACT

A device for generating soot particles with a reproducible and variable size distribution includes a combustion chamber (1), to which it is possible to supply fuel and oxidation gas and in which a flame may be formed, which is fed by the fuel and by the oxidation gas and which generates soot particles, and a soot removal conduit (3), which is coupled with the combustion chamber, in that, for example, it comprises an inlet from it, wherein the soot removal conduit in addition includes an inlet for a quenching gas. The combustion chamber and the soot removal conduit are part of a hollow space, which is capable of being decoupled from the ambient air in such a manner, that it is possible for it to be impinged by a pressure, which is different from the atmospheric pressure.

### BACKGROUND OF THE INVENTION

1. Field of the Invention

The invention is related to a device for generating soot with reproducible characteristics.

2. Description of Related Art

Soot generators, which produce soot with reproducible characteristics, are required for the calibration or adjusting of soot particle measuring instruments. Soot particle measuring instruments are utilized, for example, for the measurement of emission characteristics of internal combustion engines, in particular diesel engines.

In the document EP 1 055 877 a soot generator of this kind is described. In a combustion chamber, by means of a fuel gas, a diffusion flame producing soot particles is formed. The combustion chamber leads into a soot removal conduit, in which the soot particles are led away. The soot removal conduit includes a further inlet, into which it is possible to introduce quenching gas, by means of which combustion processes in the soot removal conduit are extinguished. With this it is achieved, that changes in the flow conditions in the soot removal conduit downstream of the further inlet do not have a significant influence on the characteristics of the generated soot particles.

A further development of this soot generator is disclosed in the document WO 2004/065494, which was published after the priority day of this protective right and is not prior art. In this soot generator, ambient air is drawn in, in that the soot removal conduit in the vicinity of the combustion chamber inlet is constricted in the manner of a venture nozzle. Through the flow of the quenching gas an negative pressure is produced, which results in a drawing-in of the air. Because with the air the combustion oxygen is drawn in from the ambient, and does not have to be blown in, a particularly compact construction becomes possible.

A further soot generator is shown in the U.S. Pat. No. 4,267,160, where fuel is mixed with air and in a pre-reactor partially combusted, whereupon it comes into a reaction space, in order to form soot.

In the case of all three disclosed soot generators there is the problem, that the characteristics of the soot particles are not independent of the position in the sense that under different ambient conditions—for example, at different altitudes—the same particle distributions are produced. This is because the soot formation process, among other things depends on the average free path length of the gas molecules. In the case of the soot generation for industrial purposes (as in U.S. Pat. No. 4,267,160), this disadvantage is not very considerable, when, however, the concern is the calibration of soot particle measuring instruments, it is of considerable importance. It would be desirable, if under differing ambient conditions, for example, at different altitudes above sea level or in case of different weather conditions, the same particle distributions would be produced. For many applications it is also necessary, that the soot produced not only has reproducible, but also adjustable characteristics, for example, with respect to size distribution. While different soot particle size distributions may be obtained, in that, for example, the composition of the fuel gas, its dilution with inert gas or its flow is varied or the fuel gas is formed by a liquid, finely vaporised fuel, additional possibilities of variation would, however, be desirable.

For the compensation of fluctuations in the atmospheric pressure, in WO 2004/065494 it is proposed to vary the constitution of the constriction and as a result the negative pressure. This procedure, however, only makes possible limited controlling of the prevailing conditions. A further reaching control would, however, be desirable.

It would also be desirable to have a soot generator available, which is also suitable for other applications than solely for the calibration of soot particle measuring instruments.

It is therefore the objective of the invention to make available a device and a method for the generation of soot particles with reproducible characteristics, which overcomes disadvantages of existing devices and methods and which should in particular make possible the production of soot with adjustable characteristics and/or which is suitable for applications, which go beyond the calibration of soot particle measuring instruments. For this purpose, the device should make possible the production of a gas, which contains soot particles with defined characteristics and in a defined quantity or concentration in the form of suspended particles.

This objective is achieved by the invention as it is defined in the claims.

### BRIEF SUMMARY OF THE INVENTION

The device includes a hollow space, in which the soot particles are produced. The hollow space is decoupled from the ambient air in the sense that the supply—and the taking away of gases (and with this also of particles suspended in them) are completely controllable. Therefore it is also possible, that the hollow space is impinged upon with a pressure differing from the atmospheric pressure. Defined gas volumes are conducted from the hollow space to the outside—for example, to a set-up of measuring instruments, or to an external chamber defined by a set-up of measuring instruments. This, in contrast to prior art makes it possible to establish both the operating parameters during the soot generation as well as the removal freely, reproducibly and independent of one another. This, in contrast to prior art, in the case of which the flow in the soot removal conduit has a direct influence on the pressure conditions in the reaction chamber (for example, the combustion chamber) and in the case of which the quantity of the removed gas containing soot may have an influence on the pressure conditions at the location of the soot generation.

The hollow space is therefore a closed system, in the case of which the gas supplies and removals are controllable.

This procedure is based, inter alia, on the insight gained, that essential characteristics of the soot particles critically depend on the pressure prevailing in the combustion chamber. Thus, for example, the average particle size may rapidly vary by a factor of 1.5 to 2 as a result of pressure changes in the order of magnitude of 100 mbar. If so required, it is therefore possible to vary the pressure in the hollow space for the systematic variation of the particle size distribution and if necessary also other particle characteristics, such as the standard deviation of their size.

In accordance with a preferred first embodiment, the hollow space contains a combustion chamber, in which in an as such known manner a soot generating flame, for example, a diffusion flame may be maintained. Suppliable to the combustion chamber are fuel and oxidation gas, by which the flame is fed and in which on the basis of a local lack of oxygen also, the soot is produced. The hollow space according to this embodiment furthermore comprises a soot removal conduit, which is coupled with the combustion chamber in that it, for example, comprises an inlet from it, wherein the soot removal in addition comprises an inlet for a quenching gas.

In accordance with a second preferred embodiment, in the hollow space the soot is produced by pyrolysis. Supplied to the hollow space is a mixture of fuel and carrier gas (it is also possible, that the mixing takes place in the hollow space itself), wherein the hollow space is heated in a suitable manner, for example, by means of an electric heating of the wall of the hollow space. The carrier gas, in preference, is free of molecular oxygen or lean in oxygen. It may consist, for example, of argon, a different inert or noble gas, depending on the pyrolysis temperature also of molecular oxygen or of a mixture of these gases. Under consideration as fuel are hydrocarbons, which at room temperature are gaseous or also liquid, for example, toluene. If the fuel is liquid at room temperature, it is nebulized or evaporated outside the hollow space prior to being brought into the hollow space. In the course of the pyrolysis, the carbon lumps together into primary particles, which subsequently further grow through coagulation. The size of the soot particles, apart from the parameters already mentioned above, pressure and fuel concentration and—composition, of course also depends on the temperature in the hollow space as well as on the duration, during which the carbon atoms are subjected to this temperature. In order to control the latter, it is possible in an as such known manner after a certain time (or, in a flow through arrangement, after a certain distance) to add a quenching gas, by which, inter alia, the gas temperature is abruptly lowered.

The soot generation in the hollow space may also be effected by a combination of pyrolysis and combustion, for example, in that in a first chamber of the hollow space by means of a partial combustion (i.e., a combustion with a lack of oxygen) the gas is heated up and thereupon—still containing the residual fuel—is conducted to a second chamber, in which no oxygen is present and in which the pyrolytic processes are continued.

Finally it is also possible, that the soot particles are generated in the hollow space by the dispersion of soot powder or by another known or still to be developed method.

In accordance with a preferred embodiment—in any type of soot generation—it is possible to freely adjust the pressure in the hollow space within a range and control it. In doing so, with the other parameters kept constant, for example, with the flame kept constant, one is able to exploit the existing association between pressure and size distribution. This, for example, with the help of a table or a characteristic function (or something similar) quantitatively reflecting this association. In this manner, it is possible to adjust a certain, desired particle size distribution. This manner of controlling the soot particle characteristics is to be preferred to a variation by means of changed flame characteristics in general.

Thus, for example, the system may be designed in such a manner that it is possible to adjust the pressure within a range of 200 mbar under-pressure (in comparison with the atmospheric pressure) up to 500 mbar over-pressure. Greater pressure differences, however, are also possible.

Instead of a controlling of the pressure, one may also simply measure the pressure in the hollow space and on the basis of the table or characteristic function by a calculation correct the influence of the produced size distribution on the measurement to follow (calibration, filter test, etc.).

The approach according to the invention with a not open removal outlet of the soot conduit makes it possible, that the soot generator is also available for new applications. For example, when testing or checking filters or filter elements, the test gas has to be impinged with pressure, in order for it to flow through the filter/the filter element; apart from this, this pressure possibly is not constant during a test cycle, if the flow is to be held constant and the filter resistance in view of the accumulation of dirt contamination increases over the course of time. For these reasons, soot generators up until now were hardly under consideration for tests as well as for the quality checks of filters. Instead of this, internal combustion engines had to be utilized as soot generators, which is disadvantageous for various reasons. The approach in accordance with the invention makes it possible that a soot generator with a flame is utilized, wherein in the combustion chamber a constant pressure not necessarily corresponding to the atmospheric pressure prevails.

In case of many applications it is essential, that during the transfer of the gas containing soot from the hollow space to the ambient, the size distribution and further characteristics of the soot particles are not impaired. During the transfer through conventional valves, for example, it is possible that larger soot particles are lost through impaction and small soot particles through diffusion—wherein apart from this the valves are rapidly contaminated with dirt and become unreliable. The device for this reason is equipped with an installation for transferring defined volumes of gas containing soot from the hollow space to the ambient. According to a preferred embodiment, this is based on the following principle: A certain volume, which is small in comparison with the volume of the hollow space, is closed-off and transported to the ambient in a closed chamber, where it, for example, is passed to a conduit leading away. Subsequently the procedure is repeated as many times as required. Essential in this embodiment is the fact, that in doing so a complete pressure equalization between the hollow space and the ambient can never take place, i.e., that the gas volume of the hollow space is closed-off before it comes into contact with the external chamber.

Installations, which make this possible, are already known as such. An embodiment of an installation of this kind based on already implemented technology is a so-called rotation diluter (or rotating disc diluter). In the case of a diluter of this kind, a disc equipped with cavities transfers small volumes of raw gas to the ambient (a rotation diluter—type MD19-2E—is available from the assignee, Matter Engineering AG; corresponding information is to be found in Ch. Hueglin, L. Scherrer and H. Burtscher, J. Aersol Sci. 28, p. 1049 (1997) or directly from the manufacturer). As an alternative, it is also possible to utilize rotating cylinders with pistons moving to and fro or other installations corresponding to the above principle, for example, with closed-off volumes transferred in a linear manner instead of through a rotation.

The selection of the installation mentioned for the transfer of gas containing soot from the hollow space to the ambient is independent of the kind of soot generation. In accordance with an alternative embodiment, the installation contains a critical nozzle (i.e., a nozzle with flow in the supersonic range), a needle valve or a tight opening in preference adjustable with regard to its size, of the type of an iris diaphragm. Nozzles or needle valves are suitable for soot particles in the sub-micrometer range, which in a flow behave practically like gas particles, for which reason there is hardly any tendency for impaction. On the basis of the high speeds in nozzles/valves of this kind, also a significant loss of particles through diffusion on an adsorbing wall is hardly to be observed.

As an additional preferred characteristic—in the case of soot generation by means of a flame—it is possible that in the hollow space an ignition device for igniting the flame is provided. It has become manifest, that an ignition device of this kind may also be arranged outside the combustion chamber in the soot removal conduit or in the fuel—or oxidation gas supply lines. Before the ignition, the naturally occurring diffusion ensures that also in these cases in the vicinity of the ignition device a sufficiently high concentration of fuel and oxidation gas for igniting the flame is present. The arrangement of the ignition device outside the combustion chamber is even to be particularly preferred, because the flow conditions in the combustion chamber should, if possible be left uninfluenced, in order that in its environment no turbulent flows are produced which could endanger the reproducibility of the soot generation.

### BRIEF DESCRIPTION OF THE DRAWINGS

In the following, examples of embodiments of the invention are described in detail on the basis of drawings. These drawings illustrate:

FIG. 1 a schematic, sectional side view of an example of an embodiment of the invention.

FIG. 2 a detail from FIG. 1, wherein possible arrangements of an ignition device are indicated.

FIG. 3 very schematically an alternative installation to the rotation diluter for the transfer of gas containing soot from the inside to the ambient.

FIG. 4 schematically a filter checking installation.

FIG. 5 also schematically a filter checking installation.

FIG. 6 a calibration arrangement for a Constant Volume Sampler CVS.

FIG. 7 an alternative example of an embodiment of the invention, also as a schematic illustration.

### DETAILED DESCRIPTION OF THE INVENTION

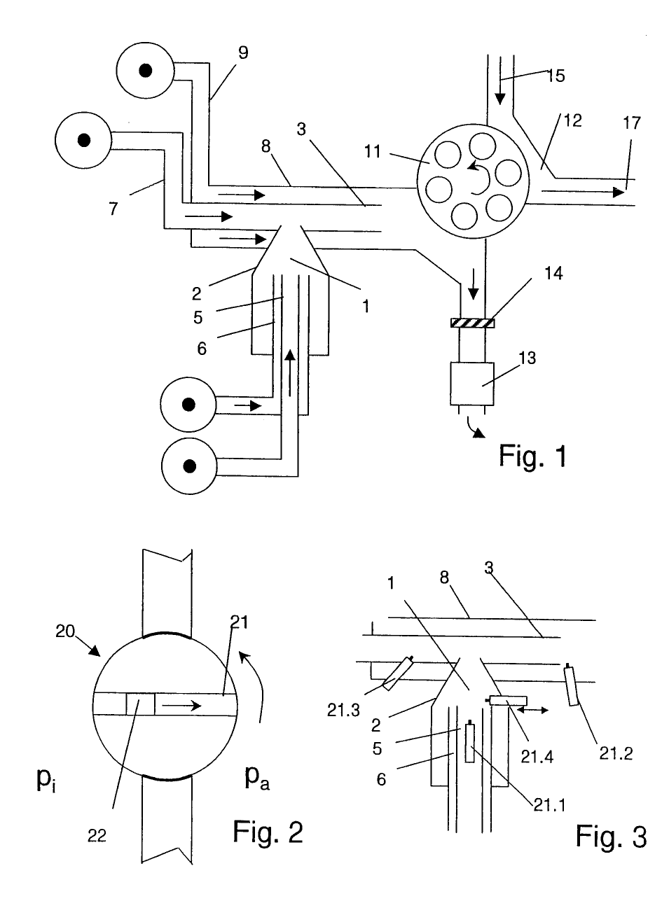

The device illustrated in FIG. 1 comprises a burner with a combustion chamber 1, which leads into a soot removal conduit 3 extending approximately perpendicularly to the combustion chamber housing 2. Leading into the combustion chamber are a fuel (in preference fuel gas) supply line 5 and a oxidation gas supply line 6 each extending approximately vertically, wherein the fuel supply line and the oxidation gas supply line, for example, are arranged coaxially. This arrangement with fuel gas and oxidation gas supplied to the location of the foreseen flame is utilized for diffusion flames, in the case of which the fuel gases and oxidation gases only mix in the flame through diffusion. The invention is equally well suitable for other types of flame, for example, pre-mixed flames, to which an already mixed fuel—/oxidation gas mixture is supplied.

Leading into the soot removal conduit 3 is a quenching gas supply line 7. Utilized as quenching gas, for example, is a chemically inert gas, such as nitrogen or a noble (inert) gas. The utilization of air is also possible, because on the basis of the high activation energy of carbon compounds, combustion or conversion processes are stopped solely on the basis of the cooling effect of the quenching gas. The soot removal conduit 3 has an open conduit end and extends coaxially to a jacket tube 8, into which a dilution gas line 9 leads. The burner in this embodiment is on the whole similar to that in the document EP 1 055 877 A, in particular to the burner described in column 5, line 27, column 9, line 44. With respect to the construction and the operating principle of the burner as well as with regard to the utilisable fuels, oxidation gases, quenching gases and gases to be admixed to these and dilution gases, reference is expressly made here to this document, wherein in the case of the device described here the burner outlet opening is replaced by other installations described in the following.

Arranged in the flow direction below the open end of the soot removal conduit 3 along the chamber or following the chamber formed by the continuation of the jacket tube 8 (the distances in the drawing are not represented to scale) is a rotation diluter 11, which is only very schematically illustrated in the Figure. With it, small volumes of the gas containing soot present in the removal conduit are transferred from the latter to a measuring line 12. In doing so, the volumes transferred are pneumatically decoupled from the removal conduit. Optionally it is possible, that the measuring line in addition to the transferred gas containing soot is supplied with dilution measuring carrier gas; the corresponding gas flow is depicted indicated by an arrow 15. Following the measuring line 12, resp., the outlet 17 of the device, measuring—and testing set-ups may be arranged, as will still be explained further below.

Gas not transferred with the rotation diluter is removed through a branch of the removal conduit or through a throttle element 13. A filter element 14 may be connected ahead in series with the throttle element, in order that the throttle element 13 does not get contaminated with soot rapidly. Other designs of the outlets are conceivable of course and advantageous depending on the specification; for example, it is possible that the removal conduit branches out ahead of the rotation diluter, wherein only a (smaller) part of the gas containing soot, for example, is conveyed past the rotation diluter by a small feed pump with a filter connected ahead in series through a branch of the removal conduit, while the other part of the gas is taken away through the other branch of the removal conduit through a filter 14 and throttle element 13.

The combustion chamber, the soot removal conduit, the jacket tube enveloping it, the removal line (up to the rotation diluter, resp., the throttle element) as well as the supply lines together form a hollow space, which is closed-off to the outside. For the operation, for example, the flow of the supplied combustion—, oxidation—, quenching—and dilution gas volumes are adjusted in such a manner, that an optimum soot generation with respect to the quantity of the generated soot and the laminarity of the flow is produced. Thereupon, on the basis of data determined at an earlier point in time, an optimum operating pressure for the required particle size distribution is selected. The gas volume taken out through the throttle element 13 is controlled by means of a control system in such a manner, that the required operating pressure is produced. A (not depicted) pressure sensor in the hollow space provides the data about the pressure necessary for this. The controlling of the soot quantity takes place on the one hand if so required through the dilution ratio, on the other hand through the quantity of gas transferred with the rotation diluter 11. The calculation of the optimum pressure and the controlling of it may take place manually, for example, with the help of a table or a characteristic curve. It is also possible, however, to carry out the determination and/or control of the pressure electronically, for example, with the help of a computer with user interface.

It has become manifest, that in the case of hydrocarbon gases (for example, propane) as fuel and dried and filtered ambient air as oxidation gas, particle size distributions with average particle sized of between 30 nm and 250 nm may be achieved, wherein the particles become bigger the higher the pressure within the combustion chamber is. A comparatively moderate pressure increase of around 100 mbar is possibly already sufficient to increase the average particle size from 50 nm to 75 nm. Particle concentrations obtained at the outlet side of the device amount to, for example, between 10⁷ cm⁻³ and 10⁹ cm⁻³.

Instead of the rotation diluter, it is also possible that other installations for the transfer of gas from the hollow space in a (measuring-) line decoupled from the hollow space are present. FIG. 2 shows a very schematic illustration of a possible principle. The dilution device 20 illustrated there between the hollow space (pressure: pᵢ) and an external chamber (pressure: pₐ) has a rotating cylinder 21 with a piston 22 displaceable within the cylinder between stops. After each rotation of the cylinder by 180°, the piston is moved along the cylinder from one stop to the other, wherein the volume contained in the cylinder is expelled on the one side and simultaneously the cylinder on the other side fills up again with gas containing soot. When pᵢ>pₐ, the displacement of the piston takes place automatically on the basis of the pressure difference, if not, then a drive mechanism for the piston has to be provided. The controlling of the transferred quantity takes place through the speed of rotation of the cylinder. The specialist will know or be capable of conceiving many other such mechanisms, which make possible the transfer of a gas volume from one vessel into another vessel decoupled from it.

Still another embodiment provides for the utilization of a critical nozzle or of a needle valve (not illustrated), with which the gas containing particles is transferred to the ambient with a high speed. Also the utilization of a small opening, for example, the opening of an iris diaphragm, through which gas containing particles flows out, is possible.

In an essentially closed-off system, the question about the ignition of the flame possibly arises. While systems for the automatic, for example, electrically operated ignition of gas flames as such have already been known for a long time, here, however, the additional problem arises, that the laminarity of the gas flow in the flame, in its immediate vicinity and wherever combustion—and coagulation processes or other processes influencing the characteristics of the soot particles take place, has to be ensured. For this reason, in general it is not possible to place a conventional spark plug at the position of the flame, and systems in accordance with prior art therefore refrain from using installations for the electrically controlled ignition of the flame.

In accordance with an embodiment of the invention, the device comprises an ignition device for igniting the flame in the closed-off hollow space. The ignition device is in preference arranged outside the combustion chamber. Four examples of possible arrangements are indicated in FIG. 3. It goes without saying, that in reality, in general, not as illustrated in FIG. 3, all four ignition devices are present, but preferably just one of them.

A first possible arrangement of an ignition device 21.1 is inside the fuel or oxidation gas supply line. Also at the outlet of the soot removal conduit 3, where the combustion product containing soot and mixed with quenching gas is mixed with the diluting gas, it is possible for an ignition device 21.2 to be arranged. A further possible arrangement of an ignition device 21.3 is inside the quenching gas supply line, upstream of the mouth of the combustion chamber 1. The ignition device may protrude into the hollow space from the outside through correspondingly provided openings, as is illustrated in two cases (corresponding to the devices 21.2 and 21.3) and it is possible to electrically actuate it in accordance with the principle of a spark plug. Alternatively, it may also be based on a catalytic principle and, for example, comprise a catalytically active large platinum surface. In all cases illustrated, the ignition device is arranged away from the combustion chamber and in such a manner, that in the zone, in which soot particles are generated and where it is possible, that they change chemically or physically (coagulation), the laminar flow is not impaired. Nonetheless it has become manifest, that an ignition is possible on the basis of the diffusion processes created prior to the combustion.

Alternatively to the arrangements described above, it is also possible to provide a mechanically displaceable ignition device. A corresponding example is the fourth illustrated ignition device 21.4. This ignition device 21.4 protrudes into the combustion chamber 1 through an opening in it. For the ignition, it is moved into a position corresponding approximately to the illustrated one. Subsequently, for a trouble-free operation it is retracted to such an extent, that the flows in the combustion chamber are laminar once more.

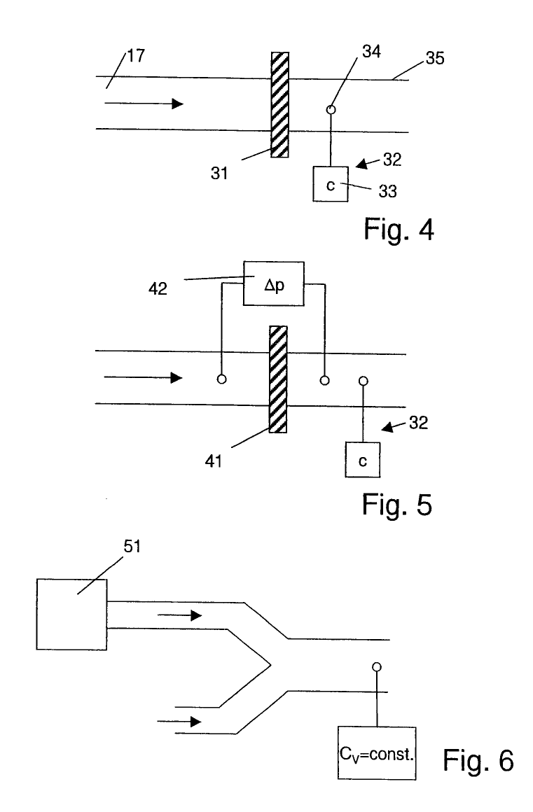

FIG. 4 illustrates a filter testing—or filter control arrangement, as it may be utilized for the quality control in the filter manufacturing industry. The outlet 17 of a device of the type described above is connected with the filter 31 with the measuring line. In the direction of flow behind the filter 31 a measuring arrangement 32—here a measuring device 33 with measuring sensor 34—is attached. On the basis of the pressure drop produced by the filter, in case of an open outlet of the measuring tube 35, the pressure is higher than the atmospheric pressure and with a changing volume flow or with changing filter characteristics it is not constant. The procedure in accordance with the invention, despite this, permits the injection into the arrangement of soot in a known, usually small, reproducible quantity with known, reproducible characteristics (particle size and/or—composition). The particle quantity detected by the filter arrangement and the particle characteristics are then characteristic for the filter properties. Measuring arrangements for the determination of particle quantities and particle characteristics, based on the measurement of the mobility or on optical, photoelectric, gravimetric or other principles are as such known and will not be explained in detail here; reference is made to the comprehensive specialist literature.

The arrangement according to FIG. 5 serves for checking filters, as it, for example, is utilized during the development of filters. Test filters 41 are subjected to an extensive test, wherein the filter is also subjected to greater, always, however, reproducible quantities of soot. In addition to a measuring arrangement 32 for the particle characteristics, a pressure measuring device 42 is indicated, which measures the pressure drop over the test filter 41—in function of the volume flow and possibly of the amount of soot filtered out up until then. The volume flow through the test filter may be varied over the course of the complete testing process, and it is possible, for example, that it attains comparatively high values of up to 1.5 m³/min or more. The volume flow of gas containing soot admixed to it by the dilution device—in this embodiment preferably a critical nozzle, a needle valve or an iris diaphragm—amounts to, for example, a flow variable between 0 and 30 l/min. The overall volume flow, controllable by controlling the carrier gas volume in the measuring line 12, and the quantity of soot therefore may be adjusted independently of one another; equally, the size distribution of the soot particles may be controlled independently of the two volume flows. All this becomes possible on the basis of the pneumatic decoupling of the hollow space of the soot generation in accordance with the invention.

It is also possible to utilize the device according to the invention, following a scaling up to larger dimensions and greater power, for the calibration of complete installations—this in contrast to individual measuring apparatuses—, for example, of so called Constant Volume Samplers' (CVS). CVS—installations are utilized for the checking of emission characteristics of internal combustion engines in function of the load. With them, the particle emission in function of the motor performance, therefore, for example, of the kilometers traveled or of the kilowatt hours produced, are determined. In a CVS a varying particle containing exhaust gas flow is mixed with dilution gas in such a manner, that the resulting volume flow is constant. The particle concentration in the volume flow is then a measure for the overall particle emission quantity.

In accordance with one aspect of the invention, a device according to the invention for the generation of soot within a combustion chamber and a soot generating flame in it is connected with the inlet of an installation of this kind instead of the internal combustion engine or a vehicle. The approach in accordance with the invention in case of a flame burning constantly of a—correspondingly large dimensioned—device 51 of the kind according to the invention enables the generation of a gas flow with varying particle quantity and if so required particle concentration, wherein the particle size distribution is independent of the quantity. If one would like to simulate the emission of an internal combustion engine, then it possibly is necessary to inject the gas flow containing soot into the installation with pressure, as is also the case with an internal combustion engine operating under load. This too is only possible with the decoupling in accordance with the invention of the hollow space of the device and the ambient.

The utilization of a soot generator with a flame for the calibration or testing of large installations for the measuring of emissions is a further aspect newly added by the invention.

The embodiments of the invention described above are solely examples and may be changed in many ways. Thus, for example, the shape of the burner is in no respect limited to the shape depicted. While a T-shaped arrangement with a vertical combustion chamber and a horizontal soot path conduit is advantageous in many respects, it is in no respect necessary. Also completely vertical arrangements with a combustion chamber, which passes over directly into an also horizontal soot conduit, are conceivable. In addition, it is possible to utilize other forms of soot generation, for example, pyrolysis. As already mentioned, also for the dilution device, or, in general, for the means for the transfer to the ambient of controlled quantities of gas containing soot many different solutions are conceivable. The only essential feature is that the combustion chamber (or soot generation chamber) is completely decoupled from the ambient.

Furthermore, apart from the pressure or instead of the pressure, additional control parameters for the adjustment of required soot particle distributions may be selected, for example, a fuel gas mixture or its dilution with inert gas, the ratio of combustion gas to oxidation gas or others. The adjustment of the pressure does not necessarily (solely) have to take place by means of a throttle element 13, but may also take place in a different manner, for example, through the quantity of the quenching gas supplied, etc.

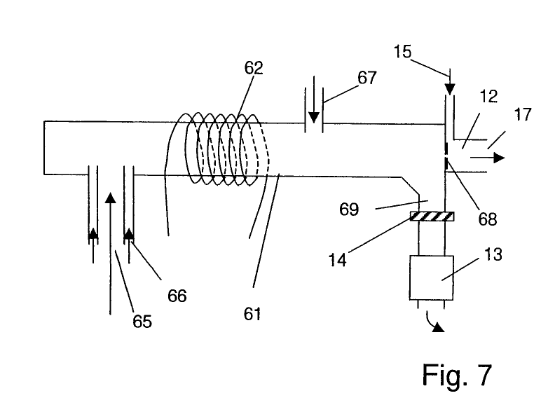

The device illustrated in FIG. 7 is foreseen for the generating of gas containing soot by pyrolysis and of transferring it to an external chamber in defined quantities. The device comprises a pyrolysis tube 61, which is manufactured out of a heat-resistant material (for example, out of molybdenum, tantalum, tungsten or ceramic materials). In place of a tube, it goes without saying that also other shapes of containers are conceivable, instead of a pyrolysis tube then in general a pyrolysis chamber is present. The device furthermore comprises means for heating up the pyrolysis tube (or the pyrolysis chamber, respectively) at least in certain zones, for example, by means of an electric heating system 62, which heats up a zone of the tube wall. Leading into the pyrolysis tube respectively are a fuel supply line 65 and a carrier gas supply line 66, wherein the fuel supply line and the carrier gas supply line in the illustrated embodiment are arranged coaxially. Utilized as fuel, for example, is a hydrocarbon gas, as carrier gas in preference a noble gas, in particular argon, also possible, however, is the utilization of other inert gases, for example, nitrogen. It is also possible, that the fuel and the carrier gas get into the pyrolysis tube pre-mixed in a common supply line. This is preferred in particular in the case of fuels, which are liquid at room temperature (for example, toluene). In this case, for example, the carrier gas will flow through the fuel before it reaches the pyrolysis tube.

In the heated zone, the fuel—carrier gas mixture heats up. The temperature of this mixture is determined by the size of the heated zone of the tube wall—as well as, if so applicable, by possible surface enlarging structures, for example, rib structures—, their temperature as well as by the flowing through speed. In the interior of the pyrolysis tube (the pyrolysis chamber), for example, a—not depicted—temperature sensor is present. It is possible, for example, to operate the device in such a manner, that the temperature of the fuel—carrier gas mixture reaches between 1000° C. and 1400° C., particularly in preference between 1100° C. and 1300° C. The C—H—bonds do not resist these temperatures, and carbon conglomerates may be formed, which coagulate into soot particles.

Leading into the pyrolysis tube 61 is also a quenching gas supply line 67. Utilized as quenching gas, for example, is a chemically inert gas, such as nitrogen or also a noble gas (for example, the same one as the carrier gas). The utilization of air is also possible, because on the basis of the high activation energy of carbon compounds combustion—or transformation processes are suppressed already solely through the cooling effect of the quenching gas. The arrangement advantageously is such, that the fuel—carrier gas mixture first flows through a heated zone of the pyrolysis tube 61, before it reaches the inlet of the quenching gas supply line.

In accordance with a variant of the described embodiment, the quenching gas supply line 67 may also be left out. In place of this, it is possible, for example, also to actively cool the pyrolysis tube or a part of the hollow space.

In the direction of flow below the inlet of the quenching gas supply line the device comprises an installation for the transferring of defined gas volumes from the hollow space to an external chamber. In the depicted example of an embodiment, it contains an iris diaphragm 68, the aperture of which is controllable and with which, for example, little soot gas clouds activated in pulses by control means (not depicted) may be output to the ambient. In the example illustrated, the defined volumes are transferred to a measuring line 12. Optionally, it is also possible for the measuring line, in addition to the transferred gas containing soot, to be supplied with a diluting measuring carrier gas (this may be air); the corresponding gas flow is depicted indicated by an arrow 15. Following the measuring line 12, or the outlet 17 of the device, as in the example presented, it is possible that measuring arrangements or test arrangements are arranged.

Not transferred gas is expelled through a throttle element 13 with filter elements ahead of it. Also, in the case of this example of an embodiment, other designs of the outlets are conceivable.

The pyrolysis tube including the strand 69 leading away to the throttle element as well as the supply lines 65, 66, 67 together form a hollow space, which is closed-off to the ambient. The closed-off hollow space may be impinged with the required pressure, and through the installation (here: iris diaphragm 68) for the transferring of gas volumes defined quantities of gas containing soot may be transferred from the hollow space to a required outer chamber (here: measuring line 12).

Also in this example of an embodiment, for the operation, for example, the flow of the supplied quantities of fuel—, carrier—, quenching—and dilution gas are adjusted in such a manner, that an optimum soot formation with respect to the quantity of the generated soot and the laminarity of the flow is produced. Thereupon, on the basis of data elicited at an earlier point in time, an optimum operating pressure for the required particle size distribution is selected. Also, for the example of an embodiment illustrated in FIG. 7 with a soot formation based on pyrolysis, the size distribution of the particles produced depends on the pressure in the hollow space, because the processes taking place there (in particular the coagulation) are influenced by the average free path length in the gas.

The gas quantity expelled through the throttle element 13 is therefore controlled in such a manner by control means, that the required operating pressure is produced. A (not depicted) pressure sensor in the hollow space provides the data about the pressure necessary for this. The controlling of the soot quantity, on the one hand if so required takes place through the dilution ratio, on the other hand through the gas quantity transferred to the ambient. The achieving of the optimum pressure and the controlling of it may take place manually, for example, with the help of a table or of a characteristic curve. It is, however, also possible to implement the determining and the controlling of the pressure electronically, for example, with the help of a computer with user interface.

For all examples of embodiments an operation is also conceivable, in the case of which the internal pressure is not controlled and maintained constant, but is solely measured. The dependence of the particle distribution on the pressure may be corrected by calculation, for example, also with the help of a characteristic curve or table, or with the help of an implicitly or explicitly known function. Although this embodiment does not comprise the advantages of an operation at constant pressure and therefore requires an increased calculation performance for the evaluation and possibly also possesses further uncertainties, it nonetheless also has the advantage that defined gas quantities with defined characteristics may also be transferred from the hollow space into a measuring arrangement decoupled with respect to pressure.

In deviation from the described examples of embodiments, it is also possible for the approach in accordance with the invention to be extended to further methods of soot generation in a hollow space, for example, as mentioned above, to combinations of (under stoichiometric) combustion and pyrolysis, if so required locally separated into different chambers. Also conceivable are the dispersing of soot powder or other physical and/or chemical processes.

1. A device for the generation of soot with defined characteristics for measuring and calibrating purposes, comprising a hollow space, which includes means for the generation of soot particles out of a fuel and for the production of a gas containing these soot particles, wherein the hollow space is decoupled from the ambient, so that it is capable of being impinged with a pressure differing from the atmospheric pressure, the device further comprising a means for transferring defined gas volumes from the hollow space into a measuring arrangement.

2. The device in accordance with claim 1, wherein the hollow space comprises a combustion chamber, to which the fuel as well as an oxidation gas are suppliable and in which a soot particle generating flame fed by the fuel and by the oxidation gas may be formed, wherein hollow space further comprises a soot removal conduit coupled with the combustion chamber, and wherein a quenching gas is suppliable to the soot removal conduit.

3. The device according to claim 2, further comprising an ignition device for igniting the flame in the hollow space.

4. The device in accordance with claim 3, wherein the ignition device is arranged outside the combustion chamber in the soot removal conduit, the quenching gas supply line or in the fuel gas—and/or oxidation gas supply line.

5. The device according to claim 1, wherein the hollow space is bounded by a hollow space wall and wherein means for the heating of the hollow space walls at least in zones are present, whereby the soot particles may be formed in the hollow space by pyrolysis.

6. The device in accordance with claim 5, wherein the hollow space is free of any oxygen supply lines.

7. The device according to claim 1, wherein the hollow space contains means for the dispersion of soot powder.

8. The device in accordance with claim 1, wherein the means for the transferring of defined gas volumes contains a dilution device.

9. The device according to claim 8, wherein the dilution device is a rotation diluter.

10. The device according to claim 1, wherein the means for the transferring of defined gas volumes comprises a critical nozzle, a needle valve or an iris diaphragm.

11. The device according to claim 1, further comprising control means for adjusting a controlled internal pressure.

12. A method for producing a gas with suspended soot particles with defined characteristics and in a defined quantity or concentration for measuring purposes or calibration purposes, comprising the steps of:
supplying a hollow space with a fuel gas and a carrier gas, wherein the hollow space is decoupled from the ambient, so that it is capable of being impinged by a pressure that is different from the ambient pressure;
generating soot particles in the hollow space out of the fuel suspended in a gas;
transferring defined gas volumes from the hollow space into a measuring arrangement;
and thereby producing the gas with soot particles with defined characteristics and in a defined quantity or concentration.

13. The method according to claim 12, wherein in generating the soot particles, the fuel is in part combusted in a flame in the hollow space.

14. The method in accordance with claim 13, wherein the flame is maintained in a combustion chamber, wherein the generated gas particles are conducted from the combustion chamber into a soot removal conduit, coupled with the combustion chamber, and wherein a quenching gas is supplied to the soot removal conduit.

15. The method according to claim 12, wherein for the generation of the soot particles, the fuel is mixed with a carrier gas and this is heated up in such a manner that the fuel is pyrolised.

16. The method in accordance with claim 15, wherein the carrier gas is heated up by a wall bounding the hollow space being heated at least in certain zones.

17. The method according to claim 12, wherein for the generation of soot particles suspended in a gas, soot powder is dispersed in the hollow space.

18. the method in accordance with claim 12, wherein the hollow space is impinged with a pressure different from the atmospheric pressure, and wherein this pressure is controlled.

19. A method for the testing of filters and filter elements, comprising the steps of producing a gas containing soot particles by a procedure comprising the steps of:
supplying a hollow space with a fuel gas and a carrier gas, wherein the hollow space is decoupled from the ambient, so that it is capable of being impinged by a pressure that is different from the ambient pressure;
generating in the hollow space out of the fuel soot particles suspended in a gas;
transferring defined gas volumes from the hollow space into a measuring arrangement;
and thereby producing the gas with soot particles with defined characteristics and in a defined quantity or concentration;
the method comprising the further step of conducting the gas with soot particles with defined characteristics and in a defined quantity or concentration or a mixture of the gas with soot particles with defined characteristics and in a defined quantity or concentration and at least one further gas through the filter to be tested or through the filter element to be tested.

20. The method in accordance with claim 19, wherein in the direction of flow behind the filter or the filter element, respectively, the soot concentration in the gas is measured.

21. The method according to claim 19, wherein a pressure drop produced by the filter or by the filter element, respectively in the gas flow is measured.

22. A use of a device for the production of a gas with soot particles with defined characteristics and in a defined quantity and/or concentration suspended in it for measuring purposes or calibrating purposes, with a combustion chamber decoupled from the ambient, to which fuel gas and oxidation gas may be supplied and in which a soot particle generating flame fed by the fuel and by the oxidation gas may be formed, and with a soot removal conduit coupled with the combustion chamber, wherein a quenching gas is suppliable to the soot removal conduit, for the testing of filter elements or for the calibration of constant volume flow measuring apparatuses.

23. The method according to claim 13 wherein the flame is a diffusion flame.

24. A device for the generation of soot with defined characteristics for measuring and calibrating purposes, comprising:
a hollow space, which includes a fuel supply and an oxidation gas supply, whereby a flame fed by the fuel supply and the oxidation gas supply is formable, wherein the hollow space is closed-off from the ambient, so that it supports an inside pressure differing from an atmospheric pressure,
the device further comprising a diluter capable of transferring defined gas volumes from the hollow space into a measuring arrangement, the diluter comprising at least one compartment with a defined volume, which compartment may be brought in a first state in which it is in communication with the hollow space but is closed-off from the measuring arrangement and may be brought in a second state in which the compartment is in communication with the measuring arrangement but is closed-off from the hollow space.

25. The device according to claim 24, comprising supply control means capable of maintaining the flame to be a diffusion flame.
